(19) United States
(12) Patent Application Publication
(10) Pub. No.: US 2023/0204512 A1
(43) Pub. Date: Jun. 29, 2023

(54) DETECTION PLATFORM AND METHOD FOR DETECTING ABUSED DRUGS

(71) Applicant: National Taiwan University, Taipei (TW)

(72) Inventors: Huan-Tsung Chang, Taipei (TW); Yao-Te Yen, Taipei (TW); Yu-Syuan Lin, Taipei (TW)

(73) Assignee: National Taiwan University, Taipei (TW)

(21) Appl. No.: 17/983,401

(22) Filed: Nov. 9, 2022

(30) Foreign Application Priority Data
Dec. 28, 2021 (TW) 110149207

(51) Int. Cl. G01N 21/64; G01N 33/94

(52) U.S. Cl. CPC G01N 21/6428; G01N 21/6456; G01N 33/946; G01N 2021/6432

(57) ABSTRACT

A detection platform is suitable for detecting an abused drug in a sample. The detection platform includes a sensing array, an image and transmission tool, and a remote workstation. The sensing array includes a reaction container, gold nanoclusters, carbon quantum dots, silver nanoclusters and a mixed solution after reaction with a Marquis reagent. The reaction container has a plurality of first grooves and a plurality of second grooves. The gold nanoclusters, the carbon quantum dots, the silver nanoclusters, and the mixed solution after reaction with the Marquis reagent are arranged in the corresponding first grooves and the corresponding second grooves, respectively. When the abused drug reacts with the gold nanoclusters, carbon quantum dots and silver nanoclusters in the first grooves, respectively, and the mixed solution after the abused drug reacting with the Marquis reagent is added to the second groove, a detection result is obtained.

### CROSS-REFERENCE TO RELATED APPLICATION

This application claims the priority benefit of Taiwan application serial no. 110149207, filed on Dec. 28, 2021. The entirety of the above-mentioned patent application is hereby incorporated by reference herein and made a part of this specification.

### BACKGROUND

### Technical Field

The disclosure relates to a detection platform and a detection method, and in particular, to a detection platform for detecting multiple kinds of abused drugs and a method for detecting abused drugs.

### Description of Related Art

Illicit drug abuse of synthetic cathinones, cocaine, ketamine, methamphetamine, and heroin is currently rampant and severely affects the human health in many countries. Therefore, a rapid screening tool at an anti-drug scene is necessary. Conventional method for detecting abused drugs may include gas chromatography-mass spectrometry (GC-MS), liquid chromatography mass spectrometry (LC-MS), colorimetric test kits, immunoassays, and a handheld Raman analyzer. However, equipment of GC-MS and LC-MS is costly, and the analytic process thereof is too complex to perform analysis at the anti-drug scene. Selectivity of colorimetric test kits for analytes is unfavorable, and there may be disturbance of color matrix. Although immunoassays exhibit high selectivity, immunoassays cannot be applied to synthetic abused drugs with various chemical structures. A handheld Raman analyzer is costly and is only used for analysis of a highly pure sample. Therefore, there may be a risk of false identification in the methods above. Accordingly, a detection method is currently necessary to address the issues above.

### SUMMARY

The disclosure is directed to a detection platform and a method for detecting abused drugs exhibiting high selectivity and high sensitivity to rapidly and accurately identify multiple kinds of abused drugs and effectively reduce the probability of false identification.

A detection platform of the disclosure is adapted to detect abused drugs in a sample. The detection platform includes a sensing array, an image and transmission tool, and a remote workstation. The sensing array includes a reaction container, gold nanoclusters, carbon quantum dots, silver nanoclusters, and a mixed solution after reaction with Marquis reagent. The reaction container has multiple first grooves and multiple second grooves. The gold nanoclusters, the carbon quantum dots, the silver nanoclusters and the mixed solution after reaction with the Marquis reagent are arranged in the corresponding first grooves and the corresponding second grooves, respectively. Under ultraviolet light, the gold nanoclusters have first fluorescence, the carbon quantum dots have second fluorescence, and the silver nanoclusters have third fluorescence. When the abused drug reacts with the gold nanoclusters, the carbon quantum dots, and the silver nanoclusters in the second grooves, respectively, and when the mixed solution after the abused drug reacting with the Marquis reagent is added to the second grooves, a detection result is obtained. The image and transmission tool are configured to capture an image of the detection result, be connected to the remote workstation, and then transmit the image to the remote workstation. The remote workstation is configured to analyze the image and automatically identify the abused drug.

In an embodiment of the disclosure, when the abused drug is synthetic cathinones, the abused drug may cause the first fluorescence to be quenched, the second fluorescence to be quenched, and the third fluorescence to be enhanced, and no fluorescence polymer particles are generated in the mixed solution after the abused drug reacting with the Marquis reagent.

In an embodiment of the disclosure, when the abused drug is cocaine, the abused drug does not cause the first fluorescence to be quenched. The abused drug may cause the second fluorescence to be quenched and cause the third fluorescence to be enhanced, and no fluorescence polymer particles are generated in the mixed solution after the abused drug reacting with the Marquis reagent.

In an embodiment of the disclosure, when the abused drug is ketamine, the abused drug does not cause the first fluorescence to be quenched and does not cause the second fluorescence to be quenched. The abused drug may cause the third fluorescence to be enhanced, and no fluorescence polymer particles are generated in the mixed solution after the abused drug reacting with the Marquis reagent.

In an embodiment of the disclosure, when the abused drug is amphetamine type (methamphetamine or methamphetamine), the abused drug does not cause the first fluorescence to be quenched and does not cause the second fluorescence to be quenched. The abused drug does not cause the third fluorescence to be enhanced. Fluorescence polymer particles with fourth fluorescence may be generated in the mixed solution after the abused drug reacting with the Marquis reagent.

In an embodiment of the disclosure, when the abused drug is heroin, the abused drug may cause the first fluorescence to be enhanced, the second fluorescence to be enhanced, and the third fluorescence to be enhanced. The fluorescence polymer particles with the fourth fluorescence may be generated in the mixed solution after the abused drug reacting with the Marquis reagent.

In an embodiment of the disclosure, a sensing mechanism of the gold nanoclusters for the abused drug is electron transfer. A sensing mechanism of the carbon quantum dots for the abused drug is electron transfer. A sensing mechanism of the silver nanoclusters for the abused drug is aggregation-induced enhancement, and a sensing mechanism of the Marquis reagent for the abused drug is whether to generate the fluorescence polymer particles.

A method for detecting an abused drug of the disclosure is adapted to detect the abused drug in a sample and includes the following. First, the detection platform above is provided. Next, the sample is added into the second grooves of the sensing array so that the abused drug reacts with the gold nanoclusters, the carbon quantum dots, and the silver nanoclusters, respectively. Then, the mixed solution after the abused drug reacting with the Marquis reagent is added to the second groove. With illumination of ultraviolet light, a detection result is obtained. An image of the detection result is captured by using the image and transmission tool, and the image and transmission tool is connected to the remote workstation and transmits the image to the remote workstation. The image is analyzed by using the remote workstation to automatically identify the abused drug.

In an embodiment of the disclosure, before obtaining the detection result with the illumination of the ultraviolet light, the method further includes the following. First, a control group without the abused drug is added into the first grooves of the sensing array so that the control group reacts with the gold nanoclusters, the carbon quantum dots, and the silver nanoclusters, respectively. Then, a mixed solution after the control group reacting with the Marquis reagent is added into the first grooves.

In an embodiment of the disclosure, analyzing the image by using the remote workstation to automatically identify the type of the abused drug includes the following. A background of the image is cropped, and a pixel size of a cropped image is adjusted.

In an embodiment of the disclosure, the abused drug includes synthetic cathinones, cocaine, ketamine, amphetamine type (methamphetamine or methamphetamine), or heroin. A detection limit of the detection platform for cathinone (e.g. 4-chloroethcathinone) is 2 mM. A detection limit of the detection platform for cocaine is 1.5 mM. A detection limit of the detection platform for ketamine is 0.8 mM. A detection limit of the detection platform for methamphetamine is 1.6 mM. A detection limit of the detection platform for heroin is 1.6 mM.

Based on the above, in the detection platform and the method for detecting the abused drug of the embodiments of the disclosure, by providing the gold nanoclusters, the carbon quantum dots, the silver nanoclusters, and the mixed solution after reaction with the Marquis reagent in the sensing array, the detection platform may exhibit the high selectivity and high sensitivity for identifying 4-chloroethcathinone (synthetic cathinone), cocaine, ketamine, heroin, and methamphetamine (amphetamine type). Accordingly, the detection platform and the method for detecting the abused drug of the embodiment may accurately identify multiple kinds of abused drugs and effectively reduce the probability of false identification. In addition, through connection between the image and transmission tool and the remote workstation and automatic identification, in the detection platform and the method for detecting the abused drug of the embodiment, the abused drug may be automatically and accurately identified through a computer of the remote workstation, and the disclosure may serve as a rapid screening tool at an anti-drug scene.

In order to make the aforementioned features and advantages of the disclosure comprehensible, embodiments accompanied with drawings are described in detail below.

### BRIEF DESCRIPTION OF THE DRAWINGS

FIG. 1A to FIG. 1E are flowcharts of a method for detecting abused drugs according to an embodiment of the disclosure.

FIG. 2A to FIG. 2E are transmission electron microscope (TEM) images of gold nanoclusters, carbon quantum dots, silver nanoclusters, methamphetamine-fluorescence polymer particles, and heroin-fluorescence polymer particles and relationship diagrams of particle numbers and particle sizes thereof, respectively.

FIG. 3A to FIG. 3E are charts of absorption spectra and emission spectra of gold nanoclusters, carbon quantum dots, silver nanoclusters, methamphetamine-fluorescence polymer particles, and heroin-fluorescence polymer particles, respectively.

FIG. 4A to FIG. 4D are selectivity tests of gold nanoclusters, carbon quantum dots, silver nanoclusters, and a Marquis reagent for different abused drugs and additives, respectively

FIG. 5 is a detection result of 4-chloroethcathinone, cocaine, ketamine, methamphetamine, heroin, and glucose in a sensing array.

FIG. 6A to FIG. 6B are Stern-Volmer dynamics results of 4-chloroethcathinone against gold nanoclusters and carbon quantum dots, respectively.

FIG. 6C is a Stern-Volmer dynamics result of cocaine against carbon quantum dots.

FIG. 6D is a chart of absorption spectra of 4-chloroethcathinone and cocaine and emission spectra of gold nanoclusters and carbon quantum dots.

FIG. 6E is a TEM image of silver nanoclusters containing ketamine.

FIG. 7 illustrates chemical structures of heroin, methamphetamine, ketamine, cocaine, and 4-chloroethcathinone.

FIG. 8A to FIG. 8F are sensitivity tests of a detection platform for 4-chloroethcathinone, cocaine, ketamine, methamphetamine, heroin, and glucose, respectively.

FIG. 9A to FIG. 9B are analysis results of smuggled samples using a detection platform.

### DESCRIPTION OF THE EMBODIMENTS

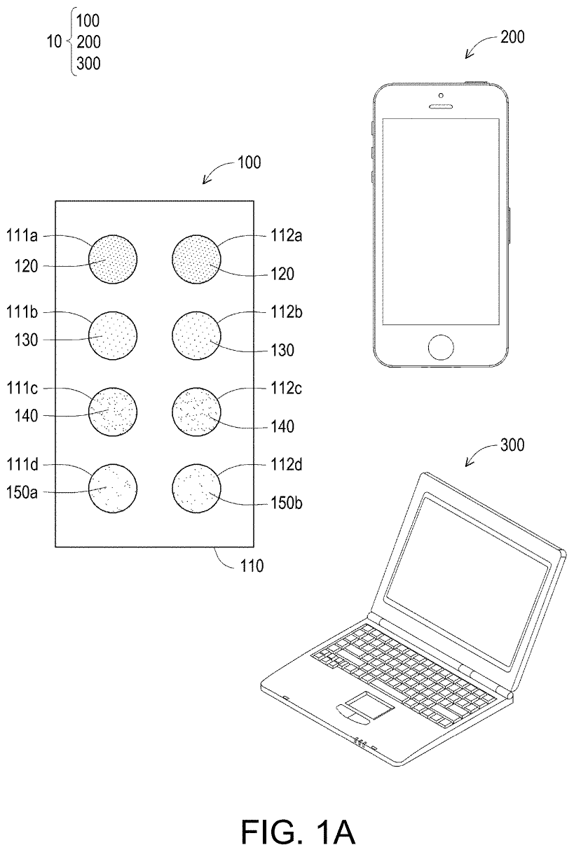

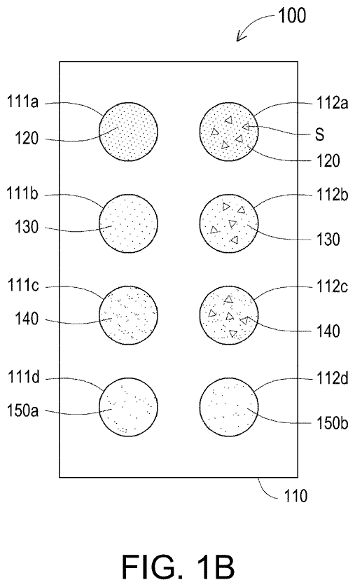

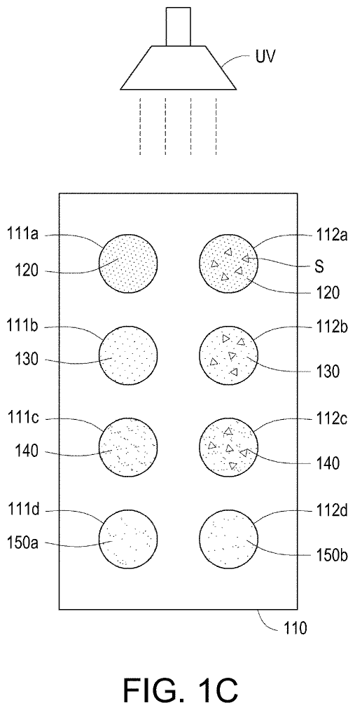

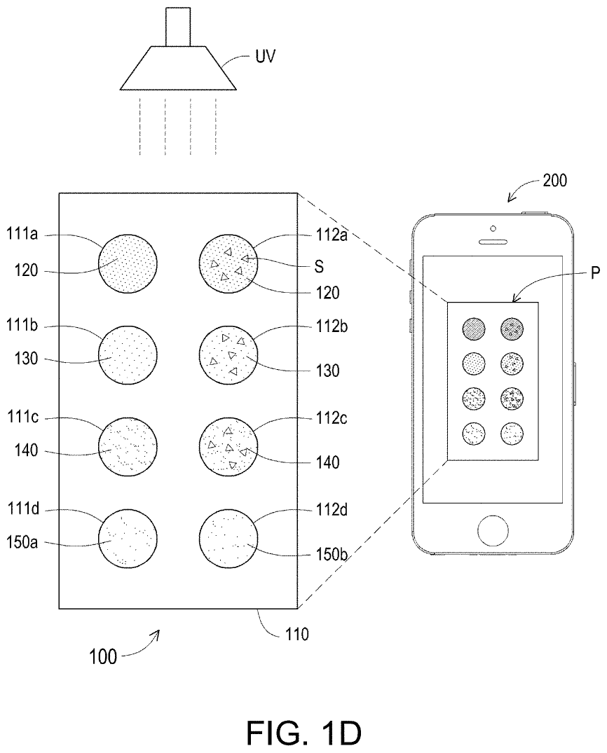

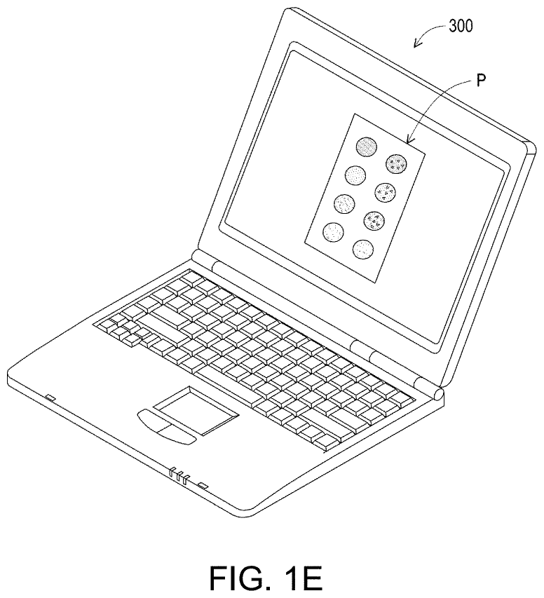

FIG. 1A to FIG. 1E are flowcharts of a method for detecting abused drugs according to an embodiment of the disclosure. A method for detecting abused drugs of the embodiment may be adapted to detect an abused drug in a sample S and includes, but not limited to, the following.

First referring to FIG. 1A, a detection platform 10 is provided. In the embodiment, the detection platform 10 may be a platform system with a deep learning power; however, the disclosure is not limited thereto. The detection platform 10 of the embodiment includes a sensing array 100, an image and transmission tool 200, and a remote workstation 300. The sensing array 100 (e.g. a 2×4 matrix illustrated in FIG. 1A) includes a reaction container 110, gold nanoclusters 120, carbon quantum dots 130, silver nanoclusters 140 and mixed solutions 150a and 150b after reaction with a Marquis reagent. The reaction container 110 has multiple first grooves 111a, 111b, 111c, and 111d (e.g. 4 first grooves illustrated in FIG. 1A) and multiple second grooves 112a, 112b, 112c, and 112d (e.g. 4 second grooves illustrated in FIG. 1A). The gold nanoclusters 120 are arranged in the corresponding first groove 111a and the corresponding second groove 112a, respectively. The carbon quantum dots 130 are arranged in the corresponding first groove 111b and the corresponding second groove 112b, respectively. The silver nanoclusters 140 are arranged in the corresponding first groove 111c and the corresponding second groove 112c, respectively. The mixed solutions 150a and 150b after reaction with the Marquis reagent are arranged in the corresponding first groove 111d and the corresponding second groove 112d, respectively.

In the embodiment, the sensing array 100 includes 8 grooves (i.e. the four first grooves 111a, 111b, 111c, and 111d and the four second grooves 112a, 112b, 112c, and 112d), and the 8 grooves are arranged in a 2×4 matrix; however, the disclosure is not intended to limit a number, a shape, and arrangement of the grooves. That is, in some embodiments, the number of the grooves may be less than or greater than 8. In some embodiments, the grooves may be arranged or spliced in a different matrix, such as a 4×4, 6×4, 2×8, 2×12, or 4×8 matrix; however, the disclosure is not limited thereto.

Next, referring to FIG. 1B, the sample S with the abused drug is added into the second grooves 112a, 112b, and 112c of the sensing array 100 so that the abused drug may react with the gold nanoclusters 120, the carbon quantum dots 130, and the silver nanoclusters 140, respectively. Then, the mixed solution 150b after the abused drug reacting with the Marquis reagent is added into the second groove 112d. In the embodiment, the sample S may be powder with the abused drug or an aqueous solution with the abused drug; however, the disclosure is not limited thereto. In an embodiment, the abused drug may include synthetic cathinones, cocaine, ketamine, amphetamine type, or heroin.

The cathinones may include 4-chloromethcathinone (4-CMC), 4-chloroethcathinone (4-CEC), 4-methylmethcathinone (4-MMC), 3-methoxy-2-(methylamine)-(4-methylphenyl)acetone-1-(mexedrone), 4-Methyl-α-ethylaminopentiophenone (4-MEAPP), 4-chloro-α-pyrrolidinopropiophenone (4-chloro-α-PPP); however, the disclosure is not limited thereto. The amphetamine type may include amphetamine and methamphetamine (MA); however, the disclosure is not limited thereto.

Next, in the embodiment, before obtaining a detection result with illumination of ultraviolet light UV, the method further includes the following. First, a control group without the abused drug is added into the first grooves 111a, 111b, and 111c of the sensing array 100 so that the control group reacts with the gold nanoclusters 120, the carbon quantum dots 130, and the silver nanoclusters 140, respectively. Then, the mixed solution 150a after the control group reacting with the Marquis reagent is added to the first groove 111d. In the embodiment, the control group may be an aqueous solution without the abused drug; however, the disclosure is not limited thereto.

Referring to FIG. 1C, with the illumination of the ultraviolet (UV) light, the detection result is obtained. Specifically, under the UV light, the gold nanoclusters 120 may have first fluorescence, the carbon quantum dots 130 may have second fluorescence, and the silver nanoclusters 140 may have third fluorescence, and fluorescence polymer particles with fourth fluorescence may be generated in the mixed solution 150b after reaction with the Marquis reagent. A wavelength of the UV light is, for example, 365 nm; however, the disclosure is not limited thereto.

In the embodiment, when the abused drug is synthetic cathinone, the synthetic cathinone may cause the first fluorescence of the gold nanoclusters 120 to be quenched, the second fluorescence of the carbon quantum dots 130 to be quenched, and the third fluorescence of the silver nanoclusters 140 to be enhanced, and no fluorescence polymer particles (FPPs) are generated in the mixed solution 150b after the synthetic cathinone reacting with the Marquis reagent.

In the embodiment, when the abused drug is cocaine, the cocaine may not cause the first fluorescence of the gold nanoclusters 120 to be quenched. The cocaine may cause the second fluorescence of the carbon quantum dots 130 to be quenched and the third fluorescence of the silver nanoclusters 140 to be enhanced, and no fluorescence polymer particles are generated in the mixed solution 150b after the cocaine reacting with the Marquis reagent.

In the embodiment, when the abused drug is ketamine, the ketamine may not cause the first fluorescence of the gold nanoclusters 120 to be quenched. The ketamine may not cause the second fluorescence of the carbon quantum dots 130 to be quenched. The ketamine may cause the third fluorescence of the silver nanoclusters 140 to be enhanced, and no fluorescence polymer particles are generated in the mixed solution 150b after the ketamine reacting with the Marquis reagent.

In the embodiment, when the abused drug is amphetamine type (amphetamine or methaphetamine), the amphetamine type may not cause the first fluorescence of the gold nanoclusters 120 to be quenched. The amphetamine type may not cause the second fluorescence of the carbon quantum dots 130 to be quenched. The amphetamine type may not cause the third fluorescence of the silver nanoclusters 140 to be enhanced, and the fluorescence polymer particles with the fourth fluorescence may be generated in the mixed solution 150b after the amphetamine type reacting with the Marquis reagent.

In the embodiment, when the abused drug is heroin, the heroin may not cause the first fluorescence of the gold nanoclusters 120 to be enhanced. The heroin may not cause the second fluorescence of the carbon quantum dots 130 to be enhanced. The heroin may cause the third fluorescence of the silver nanoclusters 140 to be enhanced, and the fluorescence polymer particles with the fourth fluorescence may be generated in the mixed solution 150b after the heroin reacting with the Marquis reagent. However, in some embodiments, fluorescence impurities in the heroin may cause the first fluorescence of the gold nanoclusters 120 to be enhanced and the second fluorescence of the carbon quantum dots 130 to be enhanced.

In addition, in the embodiment, the control group does not change intensity of the first fluorescence of the gold nanoclusters 120, the second fluorescence of the carbon quantum dots 130, and the third fluorescence of the silver nanoclusters 140, and no fluorescence polymer particles are generated in the mixed solution 150a after the control group reacting with the Marquis reagent.

Then, referring to FIG. 1D, an image P of the detection result is captured by using the image and transmission tool 200, and the image and transmission tool 200 is connected to the remote workstation 300 and transmits the image P to the remote workstation 300. In the embodiment, the image and transmission tool 200 may be, for example, a smartphone or other image and transmission tools capable of capturing an image, being connected, and transmitting the image; however, the disclosure is not limited thereto. In the embodiment, the remote workstation 300 may be a computer with a deep learning power; however, the disclosure is not limited thereto.

Next, referring to FIG. 1E, the image P is analyzed by using the remote workstation 300 and an abused drug is automatically identified by a computer of the remote workstation 300. In the embodiment, analyzing the image P by using the remote workstation 300 and automatically identifying the abused drug by using the computer of the remote workstation 300 includes, but not limited to, the following.

First, a background of the image P is cropped. Specifically, since the image P may include an image of the sensing array and the background that does not belong to the sensing array, only the image of the sensing array may be left by cropping the background that does not belong to the sensing array. Accordingly, background interference may be avoided, and an identification rate of the remote workstation 300 for the image P may be increased.

Next, a pixel size of the image P is adjusted so that the pixel size of the image P is 320×320 pixels; however, the disclosure is not limited thereto. When the pixel size is greater than 320×320 pixels, a processing time of the remote workstation 300 may be increased. When the pixel size is less than 320×320 pixels, quality of the image P and identification sensitivity of the remote workstation 300 for the image P may be reduced due to the reduced size of the image P.

Next, according to changes in fluorescence of the gold nanoclusters 120, the carbon quantum dots 130, and the silver nanoclusters 140 with the abused drug and whether fluorescence polymer particles are generated in the mixed solution 150b after the abused drug reacting with the Marquis reagent, the abused drug is automatically identified. For example, when the abused drug causes the first fluorescence of the gold nanoclusters 120 to be quenched, the second fluorescence of the carbon quantum dots 130 to be quenched, the third fluorescence of the silver nanoclusters 140 to be enhanced and no fluorescence polymer particles are generated in the mixed solution 150b after the abused drug reacting with the Marquis reagent, it may be automatically identified that the abused drug may be the cathinones.

Drawings and embodiments are provided below to illustrate the technical means adopted by the invention for achieving the purpose.

### EXPERIMENTAL EXAMPLES

### [Experimental Example 1]: Preparation of Gold Nanoclusters, Carbon Quantum Dots, Silver Nanoclusters, Methamphetamine-Fluorescence Polymer Particles, and Heroin-Fluorescence Polymer Particles

In the experimental example, a preparation method of gold nanoclusters, for example, includes, but not limited to, the following. 5 mL of a chloroauric acid (HAuCl₄) solution (10 mM) was added into 10 mL of a bovine serum albumin (BSA) solution (30 mg/mL) to form a mixed solution. After being mixed for 1 hour, 0.5 mL of a NaOH solution (1M) was added. Next, the mixed solution reacted at 45° C. for 1 hour, 0.5 mL of a NaOH solution (1M), and the color of the mixed solution turned from light yellow to brown, which meant that gold nanoclusters (BSA-Au NCs) were formed. Then, a dialysis membrane [molecular weight cut off (MWCO): 3.5 kD] was utilized, and the mixed solution was purified with ultrapure water. Three times of dialysis were conducted repeatedly within 24 hours to remove gold material that did not react. Next, the purified solution was frozen and dried to obtain light pink powder. The light pink powder was redissolved in 40 mL of a sodium phosphate buffer solution (pH 7.0, 100 mM) to prepare a gold nanocluster solution (17 mg/mL). Lastly, the gold nanocluster solution was kept at 4° C. in the dark.

In the experimental example, a preparation method of carbon quantum dots, for example, includes, but not limited to, the following. 35 mL of an L-Arginine solution (0.3 M) was added into a stainless steel container containing polytetrafluoroethylene and was heated at 240° C. for 14 hours. Next, a generated brown yellow solution was cooled to an environment temperature (approximately 25° C.). The brown yellow solution was filtered with a 0.2 mm nylon filtering membrane to remove large particles. Then, a dialysis membrane [molecular weight cut off (MWCO): 3.5 kD] was utilized, and the filtered solution was purified with ultrapure water. Three times of dialysis were conducted repeatedly within 24 hours to obtain a carbon quantum dot (C-dots) solution (0.6 mg/mL). Lastly, the carbon quantum dot solution was kept at 4° C. in the dark.

In the experimental example, a preparation method of silver nanoclusters, for example, includes, but not limited to, the following. 2.5 mL of a silver nitrate (AgNO₃) solution (200 mM) was slowly added into 20 mL of a mixed solution with thiosalicylic acid (TA) (100 mM). The thiosalicylic acid may be used to stablize the silver nanoclusters that were formed further, and the thiosalicylic acid was ortho-TA rather than meta-TA or para-TA. Next, the mixed solution was stirred for 1 hour at an environment temperature and in the dark to generate orange precipitate. Then, the precipitate was washed and purified by using the mixed solution of the ethanol and the ultrapure water and by centrifugation. The steps of washing and purifying were repeated three times to remove impurities that did not react. Next, the purified yellow orange precipitate was dried by using a freezing and drying system to obtain silver nanoclusters (TA-Ag NCs). Next, the silver nanoclusters (0.1 mg/mL) were dispersed in CHES buffer solutions with different pH values by using an ultrasonic processor to obtain a silver nanocluster solution. When the pH values of the CHES buffer solutions were 5.5, 7.0, 7.5, 8.0, 8.5, and 9.0, the concentration of the silver nanocluster solution was 15 mM. According to the testing condition of the experimental example, when the pH value of the CHES buffer solution was 8.0, the concentration of the silver nanocluster solution was 15 mM. Lastly, the silver nanocluster solution was kept at −20° C. in the dark.

In the experimental example, a preparation method of methamphetamine-fluorescence polymer particles and heroin-fluorescence polymer particles is, for example, but not limited to, the following. 500 μL of a Marquis reagent (mixture of formaldehyde and concentrated sulfuric acid) was added into a glass tube with 5 mg of methamphetamine powder (or heroin powder) to obtain a mixed solution. After reacting for 2 hours, 4.5 mL of a KOH solution (5M) was added to neutralize the mixed solution. Then, 5 mL of ethanol was added and K₂SO₄ precipitate was removed by centrifugation to obtain a supernatant. Next, a dialysis membrane [molecular weight cut off (MWCO): 500-1000 D] was utilized, and the supernatant was purified with ultrapure water. Three times of dialysis were conducted repeatedly within 72 hours to remove impurities. Lastly, the purified solution was dried to obtain methamphetamine-fluorescence polymer particles (MA-FPPs) (or heroin-fluorescence polymer particles, He-FPPs).

### [Experimental Example 2]: Analysis of Relationship of Particle Sizes and Particle Numbers of Gold Nanoclusters, Carbon Quantum Dots, Silver Nanoclusters, Methamphetamine-Fluorescence Polymer Particles, and Heroin-Fluorescence Polymer Particles, According to Transmission Electron Microscope (TEM) Images Thereof

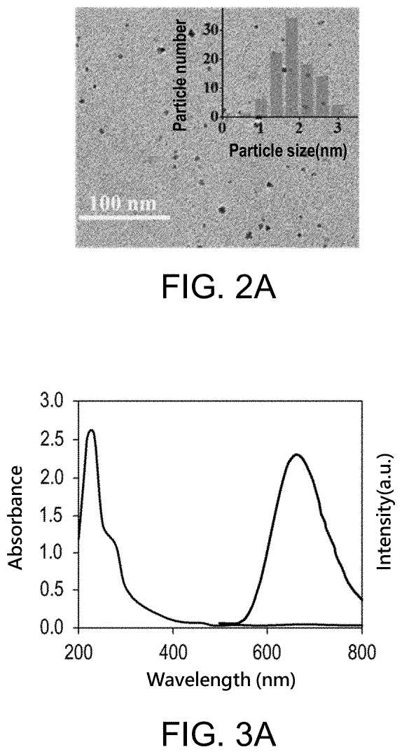

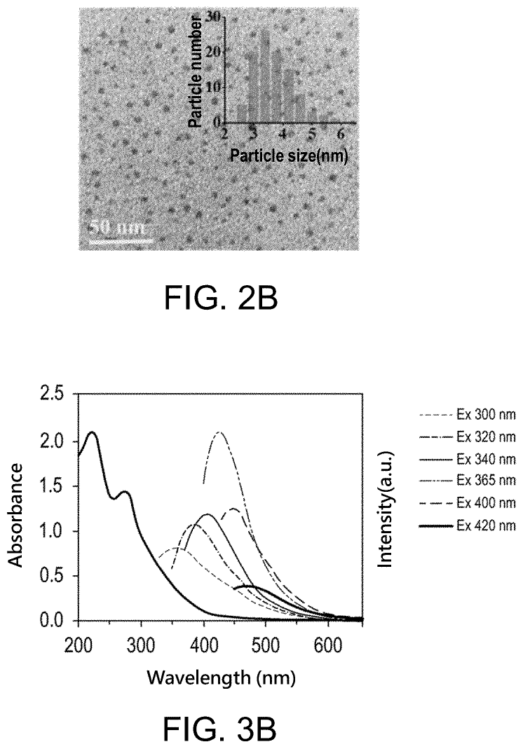

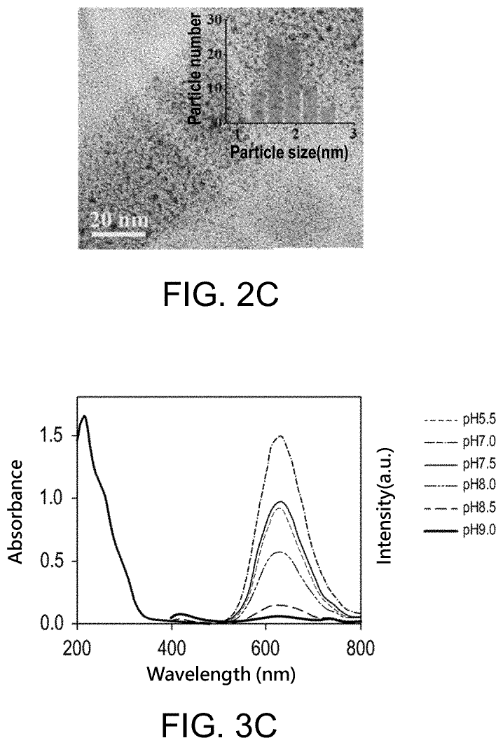

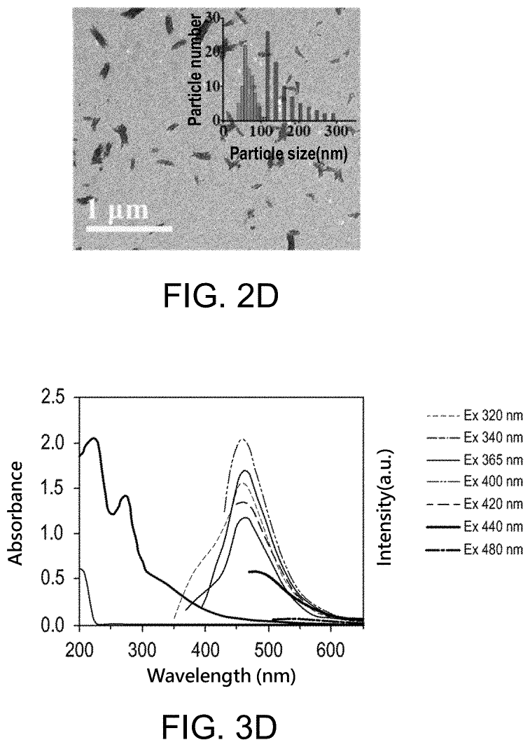

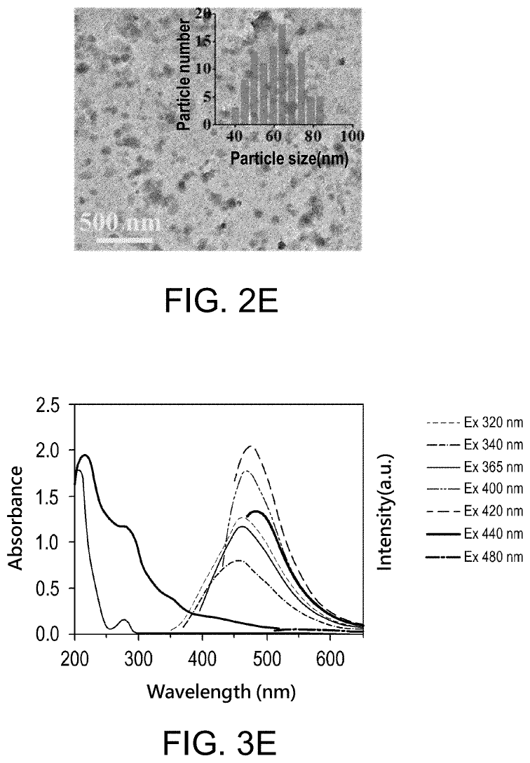

In the experimental example, gold nanoclusters, carbon quantum dots, silver nanoclusters, methamphetamine-fluorescence polymer particles, and heroin-fluorescence polymer particles were observed with a transmission electron microscope (TEM). Next, the particle sizes and the particle numbers of the gold nanoclusters, the carbon quantum dots, the silver nanoclusters, the methamphetamine-fluorescence polymer particles, and the heroin-fluorescence polymer particles were calculated, and relationship diagrams of the particle sizes and the particle numbers were illustrated. The results are shown in FIG. 2A to FIG. 2E.

According to the result of FIG. 2A, among approximately 100 gold nanoclusters, an average particle size of the gold nanoclusters was 1.8±0.7 nm. According to the result of FIG. 2B, among approximately 100 carbon quantum dots, an average particle size of the carbon quantum dots, was 3.7±0.7 nm. According to the result of FIG. 2C, among approximately 75 silver nanoclusters, an average particle size of the silver nanoclusters was 1.9±0.4 nm. According to the result of FIG. 2D, among approximately 80 methamphetamine-fluorescence polymer particles, the methamphetamine-fluorescence polymer particles were in a strip structure. An average length of the methamphetamine-fluorescence polymer particles was 160±48 nm, and an average width of the methamphetamine-fluorescence polymer particles was 63±16 nm. According to the result of FIG. 2E, among approximately 100 heroin-fluorescence polymer particles, the heroin-fluorescence polymer particles were in a spherical structure. An average particle size of the heroin-fluorescence polymer particles was 62±11 nm.

### [Experimental Example 3]: Analysis of Absorption Spectra and Emission Spectra of Gold Nanoclusters, Carbon Quantum Dots, Silver Nanoclusters, Methamphetamine-Fluorescence Polymer Particles, and Heroin-Fluorescence Polymer Particles

In the experimental example, an absorption spectrum of gold nanoclusters (85 μg/mL) in a sodium phosphate buffer solution (100 mM, pH 7.0), an absorption spectrum of carbon quantum dots (30 μg/mL) in a sodium phosphate buffer solution (100 mM, pH 9.5), an absorption spectrum of silver nanoclusters (33 μg/mL) in a CHES buffer solution (15 mM, pH 8.0), an absorption spectrum of methamphetamine-fluorescence polymer particles (0.1 mg/mL) in ultrapure water, and an absorption spectrum of heroin-fluorescence polymer particles (0.2 mg/mL) in ultrapure water were analyzed by using a ultraviolet-visible spectrophotometer. The results are shown in FIG. 3A to FIG. 3E.

In the experimental example, with excitation of ultraviolet light with a wavelength of 365 nm or other wavelength, the emission spectrum of the gold nanoclusters (85 μg/mL) in the sodium phosphate buffer solution (100 mM, pH 7.0), the emission spectrum of the carbon quantum dots (30 μg/mL) in the sodium phosphate buffer solution (100 mM, pH 9.5), the emission spectrum of the silver nanoclusters (33 μg/mL) in the CHES buffer solution (15 mM, pH 8.0), the emission spectrum of the methamphetamine-fluorescence polymer particles (0.1 mg/mL) in a sodium phosphate buffer solution (200 mM, pH 5.0), and the emission spectrum of the heroin-fluorescence polymer particles (0.2 mg/mL) in a sodium phosphate buffer solution (200 mM, pH 5.0) were analyzed by using a microplate fluorometer. The results are shown in FIG. 3A to FIG. 3E.

According to the result of FIG. 3A, the gold nanoclusters (BSA-Au NCs) had two absorbing zones between 200 nm and 300 nm, and one of the absorbing zones at 280 nm was from BSA. With the excitation of the ultraviolet light with the wavelength of 365 nm, it may be observed that the gold nanoclusters had a strong emitting zone at 680 nm, which meant that a wavelength of fluorescence emitted from the gold nanoclusters was 680 nm.

According to the result of FIG. 3B, when an excitation-wavelength of the carbon quantum dots (C-dots) was from 300 nm to 420 nm, different emission wavelengths of the carbon quantum dots were generated as the excitation-wavelength changed (excitation-wavelength dependent photoluminescence). With the excitation of the ultraviolet light with a wavelength of 365 nm, it may be observed that the carbon quantum dots had a strong emitting zone at 430 nm, which meant that a wavelength of fluorescence emitted from the carbon quantum dots was 430 nm.

According to the result of FIG. 3C, an absorbing zone of the silver nanoclusters (TA-Ag NCs) was from 200 nm to 350 nm mainly caused by TA. With the excitation of the ultraviolet light with a wavelength of 365 nm, it may be observed that the silver nanoclusters had a strong emitting zone at 630 nm, which meant that a wavelength of fluorescence emitted from the silver nanoclusters was 630 nm. In addition, fluorescence intensity of the silver nanoclusters at 630 nm may decrease as a pH value (ranging from 5.5 to 9.0) of the CHES buffer solution increased. The intensity of the fluorescence emitted from the silver nanoclusters in the CHES buffer solution (15 mM, pH 5.5) was higher, while the intensity of the fluorescence emitted from the silver nanoclusters in the CHES buffer solution (15 mM, pH 9.0) was lower.

According to the results of FIG. 3D and FIG. 3E, the absorbing peaks of the methamphetamine-fluorescence polymer particles (MA-FPPs) and the heroin-fluorescence polymer particles (He-FPP) were from 275 nm to 295 nm. With the excitation of the ultraviolet light with an wavelength of 365 nm, it may be observed that the methamphetamine-fluorescence polymer particles and the heroin-fluorescence polymer particles had a strong emitting zone at 460 nm, which meant that a wavelength of fluorescence emitted from the methamphetamine-fluorescence polymer particles and a wavelength of fluorescence emitted from the heroin-fluorescence polymer particles were 460 nm.

In addition, when an excitation-wavelength of the methamphetamine-fluorescence polymer particles and an excitation-wavelength of the heroin-fluorescence polymer particles were from 320 nm to 400 nm, the intensity of the emission wavelengths of the methamphetamine-fluorescence polymer particles and the heroin-fluorescence polymer particles may change as the excitation-wavelengths changed. However, the emission wavelengths did not change. In addition, when the excitation-wavelength of the methamphetamine-fluorescence polymer particles and the excitation-wavelength of the heroin-fluorescence polymer particles were from 440 nm to 480 nm, different emission wavelengths of the methamphetamine-fluorescence polymer particles and the heroin-fluorescence polymer particles were generated as the excitation-wavelengths changed (excitation-wavelength dependent photoluminescence).

### [Experimental Example 4]: Selectivity Tests of Gold Nanoclusters, Carbon Quantum Dots, Silver Nanoclusters, and a Marquis Reagent for Different Abused Drugs and Additives

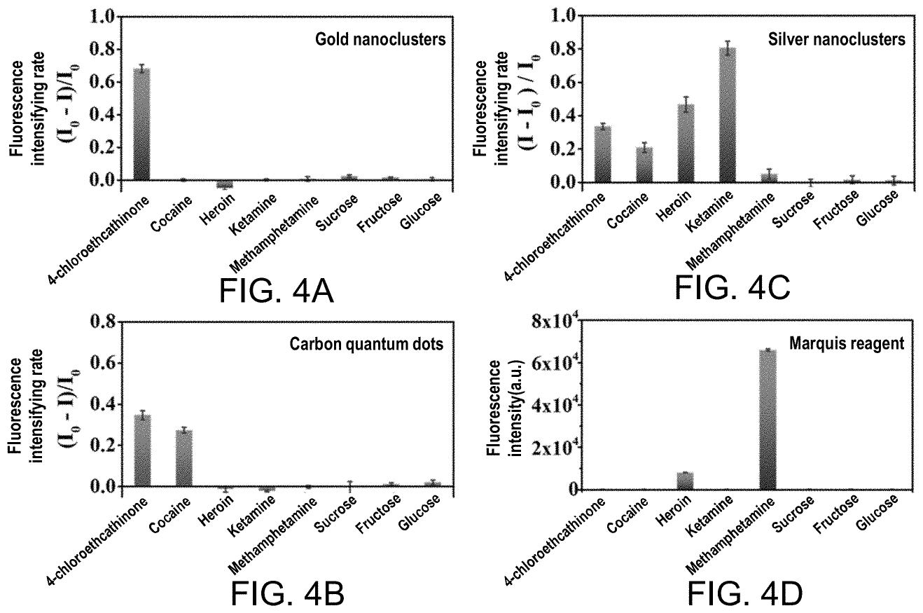

In the experimental example, an analyte was added into the first three second grooves among the four second grooves of the sensing array so that the analyte reacted with gold nanoclusters, carbon quantum dots, and silver nanoclusters, respectively. A mixed solution after the analyte reacting with a Marquis reagent was added into the fourth second groove among the four second grooves. Next, a control group without the analyte was added into the first three first grooves among the four first grooves of the sensing array so that control group without the analyte reacted with the gold nanoclusters, the carbon quantum dots, and the silver nanoclusters, respectively. A mixed solution after the control group without the analyte reacting with the Marquis reagent was added into the fourth first groove among the four first grooves. Lastly, under ultraviolet light with a wavelength of 365 nm, the intensity of fluorescence was detected, and relative fluorescence quenching rates (I₀−I)/I₀, relative fluorescence intensifying rates (I−I₀)/I₀, or fluorescence intensities were calculated. The results are shown in FIG. 4A to FIG. 4D.

In the experimental example, the analyte included abused drugs or additives. The abused drugs included 4-chloroethcathinone, cocaine, heroin, ketamine, or methamphetamine, and the additives included sucrose, fructose, or glucose. In the experimental example, I₀ is the number of the fluorescence intensity of the control group without the analyte reacting with the gold nanoclusters, the carbon quantum dots, and the silver nanoclusters, respectively, or the number of the fluorescence intensity of the mixed solution after the control group without the analyte reacting with the Marquis reagent; I is the number of the fluorescence intensity of the analyte reacting with the gold nanoclusters, the carbon quantum dots, and the silver nanoclusters, respectively, or the number of the fluorescence intensity of the mixed solution after the analyte reacting with the Marquis reagent. The concentration of all the analytes reacting with the gold nanoclusters and the carbon quantum dots was 10 mM, the concentration of all the analytes reacting with the silver nanoclusters was 4 mM, and the concentration of all the mixed solutions after the reaction with the Marquis reagent containing the analyte was 435 μM.

According to the results of the fluorescence quenching rates (I₀−I)/I₀ of FIG. 4A, the 4-chloroethcathinone may cause the fluorescence intensity of the gold nanoclusters at a wavelength of 680 nm to be quenched 68.2±2.4%, while the cocaine, the heroin, the ketamine, the methamphetamine, and the additives did not cause the fluorescence intensity of the gold nanoclusters at the wavelength of 680 nm to be noticeably quenched. That is, the gold nanoclusters exhibited the high selectivity for identifying the 4-chloroethcathinone (the cathinone). In addition, in other experimental examples, other cathinones, such as 4′-chloro-α-pyrrolidinopropiophenone, 4-chloromethcathinone, 4-methyl-α-ethylaminopentiophenone, and mexedrone, may cause the fluorescence intensity of the gold nanoclusters at the wavelength of 680 nm to be quenched 48.7±1.1%, 76.0±3.1%, 62.4±1.5%, and 39.0±0.9%, respectively.

According to the results of the fluorescence quenching rates (I₀−I)/I₀ of FIG. 4B, the 4-chloroethcathinone and the cocaine may cause the fluorescence intensity of the carbon quantum dots at a wavelength of 430 nm to be quenched 34.6±0.2% and 27.2±0.1%, respectively, while the heroin, the ketamine, the methamphetamine, and the additives did not cause the fluorescence intensity of the carbon quantum dots at the wavelength of 430 nm to be noticeably quenched. That is, the carbon quantum dots exhibited the high selectivity for identifying the 4-chloroethcathinone (the cathinones) and the cocaine. In addition, in other experimental examples, other cathinones, such as 4′-chloro-α-pyrrolidinopropiophenone, 4-chloromethcathinone, 4-MEAPP e, and mexedrone may cause the fluorescence intensity of the carbon quantum dots at the wavelength of 430 nm to be quenched 26.2±0.4%, 27.9±0.4%, 24.0±0.7%, and 28.3±0.4%, respectively.

According to the results of the fluorescence intensifying rates (I−I₀)/I₀ of FIG. 4C, the 4-chloroethcathinone, the cocaine, the heroin, and the ketamine may cause the fluorescence intensity of the silver nanoclusters at a wavelength of 630 nm to be enhanced 33±2%, 21±3%, 47±5%, and 80±4%, respectively, while the methamphetamine and the additives did not cause the fluorescence intensity of the silver nanoclusters at a wavelength of 630 nm to be noticeably enhanced. That is, the silver nanoclusters may identify the 4-chloroethcathinone (the cathinones), the cocaine, the heroin, and the ketamine.

According to the results of the fluorescence intensity of FIG. 4D, the mixed solution after the heroin reacting with the Marquis reagent may generate fluorescence polymer particles (i.e. the fluorescence intensity may be detected), and the mixed solution after the methamphetamine reacting with the Marquis reagent may also generate the fluorescence polymer particles. However, the mixed solutions after the 4-chloroethcathinone, the cocaine, the heroin, and the ketamine, and the additives reacting with the Marquis reagent did not generate the fluorescence polymer particles (i.e. the fluorescence intensity is not detected), respectively. That is, the Marquis reagent exhibited the high selectivity for identifying the heroin and the methamphetamine (the amphetamine type).

In the experimental example, the gold nanoclusters exhibited the high selectivity for identifying the 4-chloroethcathinone (the cathinones), the carbon quantum dots exhibited the high selectivity for identifying the 4-chloroethcathinone (the cathinones) and the cocaine, the silver nanoclusters exhibited the high selectivity for identifying the 4-chloroethcathinone (the cathinones), the cocaine, the heroin, and the ketamine, and the Marquis reagent exhibited the high selectivity for identifying the heroin and the methamphetamine (the amphetamine type). Therefore, the sensing array of the experimental example may accurately identify multiple kinds of abused drugs and effectively reduce the probability of false identification.

### [Experimental Example 5]: Analysis of a Detection Result of 4-Chloroethcathinone, Cocaine, Ketamine, Amphetamine, Heroin, and Glucose in a Sensing Array

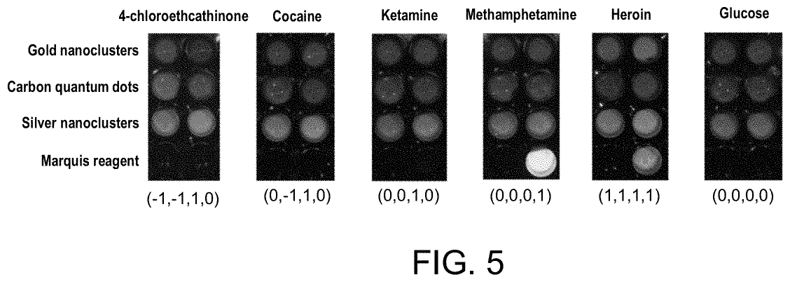

In the experimental example, an analyte was added into the first three second grooves (an experiment group) among the four second grooves of the sensing array so that the analyte reacted with gold nanoclusters, carbon quantum dots, and silver nanoclusters, respectively. A mixed solution after the analyte reacting with a Marquis reagent was added into the fourth second groove (an experiment group) among the four second grooves. Next, based on the fluorescence of the first grooves without the analyte (a control groups) or based on the fluorescence of the first groove with a mixed solution after only water reacting with the Marquis reagent (a control group), changes in the fluorescence of the second grooves with the analyte were determined. The analyte included 4-chloroethcathinone, cocaine, ketamine, methamphetamine, heroin, or glucose. Next, under ultraviolet light with a wavelength of 365 nm, the fluorescence intensity of the experiment group (the aqueous solutions with the abused drugs) and the control group (the aqueous solution without the abused drugs) was detected to obtain a detection result. Lastly, the result of changes in the fluorescence of the analyte in the gold nanoclusters, the carbon quantum dots, and the silver nanoclusters was denoted as the reference numerals (1), (0), and (−1), and the result of whether the fluorescence polymer particles were generated in the mixed solution after the analyte reacting with the Marquis reagent was denoted as the reference numerals (1) and (0). Hence, a fluorescence detection code (W, X, Y, Z) of the analyte was obtained, and the results are shown in FIG. 5.

In the experimental example, the reference numeral (1) represented that the fluorescence intensity of the second grooves (the experiment group) with the analyte was apparently greater than (i.e. a difference over 5%) the fluorescence intensity of the first grooves (the control group) without the analyte, which meant that the analyte may intensify the fluorescence. The reference numeral (0) represented that the fluorescence intensity of the second grooves (the experiment group) with the analyte was similar to (i.e. a difference below 5%) the fluorescence intensity of the first grooves (the control group) without the analyte, which meant that the analyte did not change the fluorescence intensity. The reference numeral (−1) represented that the fluorescence intensity of the second grooves (the experiment group) with the analyte was apparently less than (i.e. a difference over 5%) the fluorescence intensity of the first grooves (the control group) without the analyte, which meant that the analyte may quench the fluorescence. In addition, when the analyte was the heroin or the methamphetamine, the reference numeral (1) represented that the fluorescence polymer particles were generated in the mixed solution after the analyte reacting with the Marquis reagent, and the reference numeral (0) represented that no fluorescence polymer particles were generated.

In the experimental example, the fluorescence detection code (W, X, Y, Z) was a combination of four reference numerals. W represented whether the analyte caused the fluorescence intensity of the gold nanoclusters to be enhanced, remain constant, or be quenched. X represented whether the analyte caused the fluorescence intensity of the carbon quantum dots to be enhanced, remain constant, or be quenched. Y represented whether the analyte caused the fluorescence intensity of the silver nanoclusters to be enhanced, remain constant, or be quenched. Z represented whether the fluorescence polymer particles were generated in the mixed solution after the analyte reacting with the Marquis reagent.

According to the result of FIG. 5, since the 4-chloroethcathinone may cause the fluorescence intensity of the gold nanoclusters to be quenched (i.e. the reference numeral of W was −1), cause the fluorescence intensity of the carbon quantum dots to be quenched (i.e. the reference numeral of X was −1), cause the fluorescence intensity of the silver nanoclusters to be enhanced (i.e. the reference numeral of Y was 1), and the mixed solution after the 4-chloroethcathinone reacting with the Marquis reagent may not generate fluorescence polymer particles (i.e. the reference numeral of Z was 0), the fluorescence detection code of the 4-chloroethcathinone (cathinone) was (−1, −1, 1, 0).

According to the result of FIG. 5, since the cocaine may not change the fluorescence intensity of the gold nanoclusters (i.e. the reference numeral of W was 0), may cause the fluorescence intensity of the carbon quantum dots to be quenched (i.e. the reference numeral of X was −1), may cause the fluorescence intensity of the silver nanoclusters to be enhanced (i.e. the reference numeral of Y was 1), and the mixed solution after the cocaine reacting with the Marquis reagent may not generate the fluorescence polymer particles (i.e. the reference numeral of Z was 0), the fluorescence detection code of the cocaine was (0, −1, 1, 0).

According to the result of FIG. 5, since the ketamine may not change the fluorescence intensity of the gold nanoclusters (i.e. the reference numeral of W was 0), not change the fluorescence intensity of the carbon quantum dots (i.e. the reference numeral of X was 0), cause the fluorescence intensity of the silver nanoclusters to be enhanced (i.e. the reference numeral of Y was 1), and the mixed solution after the ketamine reacting with the Marquis reagent may not generate the fluorescence polymer particles (i.e. the reference numeral of Z was 0), the fluorescence detection code of the ketamine was (0, 0, 1, 0).

According to the result of FIG. 5, since the methamphetamine may not change the fluorescence intensity of the gold nanoclusters (i.e. the reference numeral of W was 0), not change the fluorescence intensity of the carbon quantum dots (i.e. the reference numeral of X was 0), not change the fluorescence intensity of the silver nanoclusters (i.e. the reference numeral of Y was 0), and the mixed solution after the methamphetamine reacting with the Marquis reagent may generate the fluorescence polymer particles (i.e. the reference numeral of Z was 1), the fluorescence detection code of the methamphetamine (or the amphetamine type) was (0, 0, 0, 1).

According to the result of FIG. 5, since the heroin may cause the fluorescence intensity of the gold nanoclusters to be enhanced (i.e. the reference numeral of W was 1), cause the fluorescence intensity of the carbon quantum dots to be enhanced (i.e. the reference numeral of X was 1), cause the fluorescence intensity of the silver nanoclusters to be enhanced (i.e. the reference numeral of Y was 1), and the mixed solution after the heroin reacting with the Marquis reagent may generate fluorescence polymer particles (i.e. the reference numeral of Z was 1), the fluorescence detection code of the heroin was (1, 1, 1, 1). In addition, note that the heroin may not cause first fluorescence of the gold nanoclusters to be enhanced and not cause second fluorescence of the carbon quantum dots to be enhanced. That is, the fluorescence detection code of the heroin should be (0, 0, 1, 1). However, the fluorescence detection code of the tested heroin here was (1, 1, 1, 1) because blue fluorescence impurities in the heroin may interfere the first fluorescence of the gold nanoclusters and the second fluorescence of carbon quantum dots.

According to the result of FIG. 5, since the glucose may not change the fluorescence intensity of the gold nanoclusters (i.e. the reference numeral of W was 0), not change the fluorescence intensity of the carbon quantum dots (i.e. the reference numeral of X was 0), not change the fluorescence intensity of the silver nanoclusters (i.e. the reference numeral of Y was 0), and the mixed solution after the glucose reacting with the Marquis reagent may not generate the fluorescence polymer particles (i.e. the reference numeral of Z was 0), the fluorescence detection code of the glucose was (0, 0, 0, 0).

### [Experimental Example 6]: Analysis of Sensing Mechanisms of Gold Nanoclusters, Carbon Quantum Dots, Silver Nanoclusters, and a Marquis Reagent for 4-Chloroethcathinone, Cocaine, Ketamine, Amphetamine, and Heroin

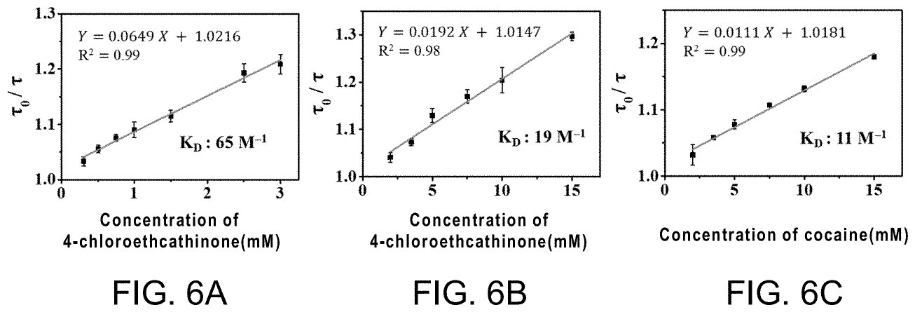

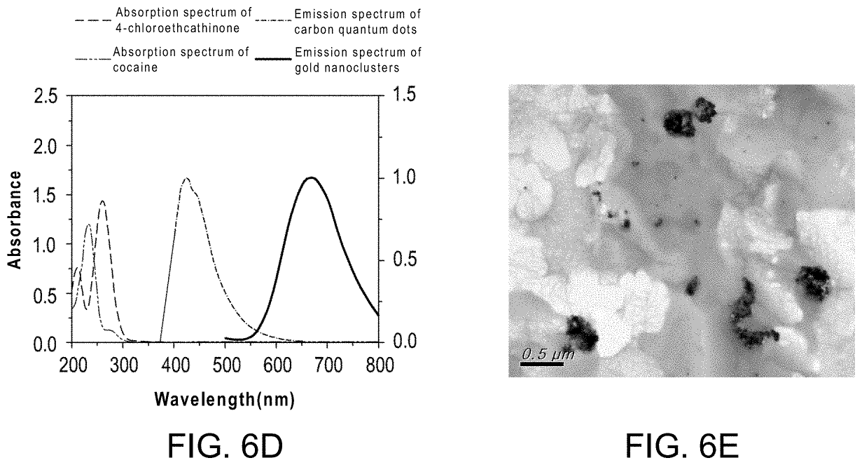

According to the results of FIG. 6A to FIG. 6C, since relations of τ₀/τ and a corresponding drug concentration of 4-chloroethcathinone against gold nanoclusters (FIG. 6A), 4-chloroethcathinone against carbon quantum dots (FIG. 6B), and cocaine against carbon quantum dots (FIG. 6C) in a dynamics Stern-Volmer equation are linear, it is shown that a sensing mechanism of the gold nanoclusters for 4-chloroethcathinone, a sensing mechanism of the carbon quantum dots for 4-chloroethcathinone, and a sensing mechanism of the carbon quantum dots for cocaine are all dynamic quenching. In addition, the dynamic quenching includes fluorescence resonance energy transfer and electron transfer. To in FIG. 6A to FIG. 6C is an original fluorescence lifetime of the gold nanoclusters or the carbon quantum dots, and T is a fluorescence lifetime of the gold nanoclusters or the carbon quantum dots after reaction with the abused drugs. Next, according to the result of FIG. 6D, since the absorption spectrum of the 4-chloroethcathinone and the absorption spectrum of the cocaine did not intersect with the emission spectrum of the gold nanoclusters and the emission spectrum of the carbon quantum dots, the fluorescence resonance energy transfer mechanism may be excluded. That is, the sensing mechanism of the gold nanoclusters for the 4-chloroethcathinone is electron transfer, the sensing mechanism of the carbon quantum dots for the 4-chloroethcathinone is electron transfer, and the sensing mechanism of the carbon quantum dots for the cocaine is electron transfer.

According to the results of the fluorescence intensifying rates (I₀−I)/I₀ of FIG. 4C in Experimental example 4, the 4-chloroethcathinone, the cocaine, the heroin, and the ketamine may cause the fluorescence intensity of the silver nanoclusters at the wavelength of 630 nm to be enhanced. Since the ketamine may cause the fluorescence of the silver nanoclusters to be enhanced the most, the ketamine is taken as an example below to describe the sensing mechanism of the silver nanoclusters for the ketamine. According to the result of FIG. 2C in Experimental example 2, the average particle size of the silver nanoclusters should be 1.9±0.4 nm. However, with the reaction of the ketamine and the silver nanoclusters, aggregation of the silver nanoclusters with the average particle size of about 0.5 μm may be generated as shown in FIG. 6E. Hence, given an increased particle size and a sensing property of enhanced fluorescence, it may be obtained that the sensing mechanism of the silver nanoclusters for the ketamine is aggregation-induced emission. Similarly, the sensing mechanism of the silver nanoclusters for the 4-chloroethcathinone is aggregation-induced emission, the sensing mechanism of the silver nanoclusters for the cocaine is aggregation-induced emission, and the sensing mechanism of the silver nanoclusters for the heroin is aggregation-induced emission.

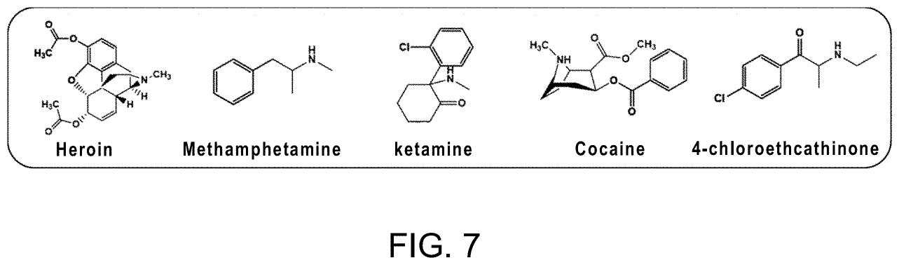

According to the results of the fluorescence intensity of FIG. 4D in Experimental example 4, the mixed solutions after the methamphetamine and the heroin reacting with the Marquis reagent may generate fluorescence polymer particles (i.e. the fluorescence intensity may be detected), respectively. However, the mixed solutions after the 4-chloroethcathinone, the cocaine, the ketamine, and the additives reacting with the Marquis reagent did not generate fluorescence polymer particles (i.e. the fluorescence intensity was not detected), respectively. Next, as shown in FIG. 7, in the overall chemical structures of the 4-chloroethcathinone, the cocaine, the ketamine, the heroin, and the methamphetamine, benzene ring substituents of the heroin and the methamphetamine are all activating substituents (i.e. substituents having electron-donating groups), and benzene ring substituents of the 4-chloroethcathinone, the cocaine, the ketamine are all deactivating substituents (i.e. substituents having electron-withdrawing groups). Next, the Marquis reagent composed of concentrated sulfuric acid and formaldehyde may generate protonated formaldehyde molecules with a positive charge. Hence, the abused drugs having benzene ring substituents that are all activating substituents (i.e. the heroin and the methamphetamine) may react with the protonated formaldehyde molecules with a positive charge generated by the Marquis reagent to generate the fluorescence polymer particles.

### [Experimental Example 7]: Establishment of a Platform System of Deep Learning Drug Detection with a Deep Learning Power

Since law enforcement officers are unable to easily or rapidly determine the changes in the fluorescence intensity according to the detection results in Experimental example 5, in the experimental example, abused drug may be automatically identified by establishing a computer and a platform system of deep learning drug detection with a deep learning power to help law enforcement officers rapidly and accurately determine a detection result.

Specifically, in the experimental example, the platform system of deep learning drug detection (i.e. the detection platform) may include a sensing array, a smartphone (i.e. an image and transmission tool), and a computer with the deep learning power (i.e. a remote workstation). The smartphone was configured to photograph the detection result of the sensing array into an image and transmit the image to the computer through the Internet connection. The computer had a YOLO v4 (You Only Look Once v4) model to perform deep learning and image recognition.

Before the platform system of deep learning drug detection was used, the platform system of deep learning drug detection had to be trained and deep learning had to be performed on the platform system of deep learning drug detection. The process of training the platform system of deep learning drug detection and performing deep learning include, for example, the following. First, the sensing array was adopted to detect five types of abused drugs (4-chloroethcathinone, cocaine, ketamine, methamphetamine, and heroin) at different concentration and one type of additive (glucose) and detection results were obtained. Next, the detection results were photographed into images by using the smartphone. Next, the images were cropped to remove the background to leave the image of only the sensing array. The pixel size of the images was adjusted to 320×320 pixels. The images were commented to mark that the images were derived from the detection results of which abused drugs. “CEC” represented cathinones, “Co” represented cocaine, “H” represented heroin, “K” represented ketamine, “MA” represented methamphetamine, and “G” represented non-abused drugs. Next, all the images marked with the detection results of the abused drugs were input into a convolutional neural network (CNN) system in the YOLO v4 model. The system may classify the images according to the comments and learn to obtain image characteristics from different types of images on its own. After the deep learning computer finished obtaining the image characteristics on its own, the platform system of deep learning drug detection of the experimental example was optimized to automatically identify the abused drug according to the images. The platform system of deep learning drug detection identified an identification code of the cathinones as “CEC”, an identification code of the cocaine as “Co”, an identification code of the heroin as “H”, an identification code of the ketamine as “K”, an identification code of the methamphetamine as “MA”, and an identification code of the non-abused drugs as “G”.

### [Experimental Example 8]: Analysis of Sensitivity (i.e. a Detection Limit) of a Detection Platform for 4-Chloroethcathinone, Cocaine, Ketamine, Methamphetamine, Heroin, and Glucose

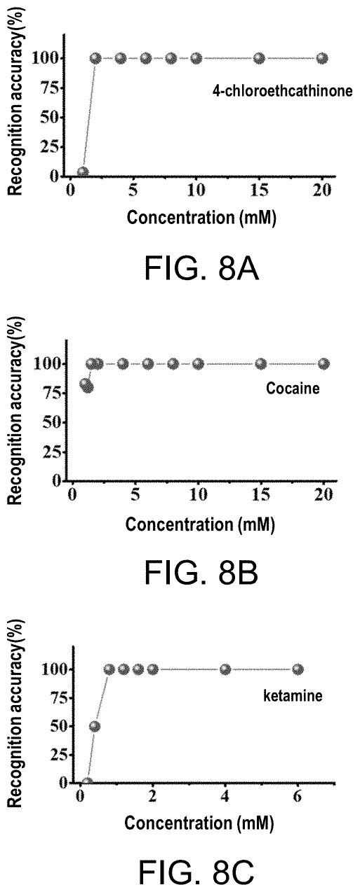

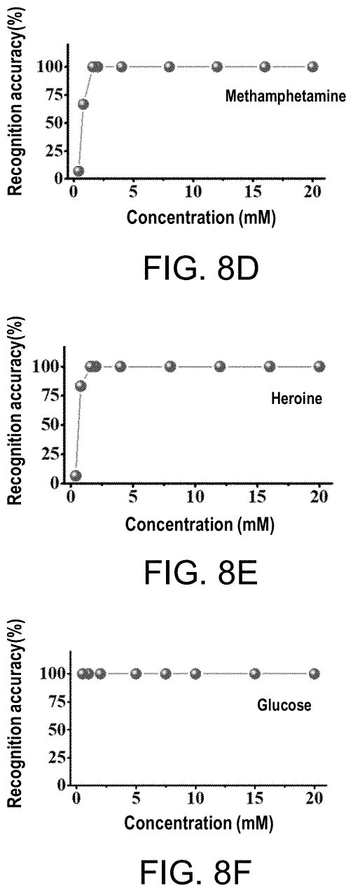

In the experimental example, analysis below was performed by using the platform system of deep learning drug detection (i.e. the detection platform) of Experimental example 6. An analyte at certain concentration was added into the second grooves of the sensing array so that the analyte at the certain concentration may react with gold nanoclusters, carbon quantum dots, and silver nanoclusters, respectively. Then, a mixed solution after the analyte at the certain concentration reacting with Marquis reagent was added to the second groove. Next, under ultraviolet light with a wavelength of 365 nm, the sensing array after reaction was detected to obtain a detection result. Fluorescence matrix images of the detection result were captured by using the smartphone, and the images were transmitted to the computer with the deep learning power. The images were cropped to remove the background to leave the images of only the sensing array. Next, after the pixel size of the images was adjusted to 320×320 pixels by using the computer, the images were input to the deep learning computer for automatic identification to obtain whether the detection platform could correctly identify the analyte at the certain concentration. Hence, the sensitivity (i.e. the detection limit) of the detection platform for the analyte may be obtained, and the results are shown as FIG. 8A to FIG. 8F.

In the experimental example, the analyte included 4-chloroethcathinone, cocaine, ketamine, methamphetamine, heroin, or glucose. The concentration of the 4-chloroethcathinone was 1.0 mM to 20 mM. The concentration of the cocaine was 1.0 mM to 20 mM. The concentration of the ketamine was 0.2 mM to 6 mM. The concentration of the methamphetamine was 0.4 mM to 20 mM. The concentration of the heroin was 0.4 mM to 20 mM. The concentration of the glucose was 0.5 mM to 20 mM. In addition, in the experimental example, ten images were captured for the detection result of each concentration of each analyte taken by each smartphone. As a result, 1560 images were captured in total. In the experimental example, a confidence threshold value of the detection platform was 0.7.

According to the result of FIG. 8A, recognition accuracy of the detection platform for the 4-chloroethcathinone at the concentration of 2.0 mM to 20 mM was 100%, while recognition accuracy for the 4-chloroethcathinone at the concentration of 1.0 mM was 3.3%. That is, the detection limit of the detection platform for the 4-chloroethcathinone (or cathinones) was approximately 2.0 mM, and the detection platform exhibited high sensitivity for identifying the 4-chloroethcathinone (or the cathinones).

According to the result of FIG. 8B, recognition accuracy of the detection platform for the cocaine at the concentration of 1.5 mM to 20 mM was 100%, while recognition accuracy for the cocaine at the concentration of 1.0 mM was 83.3%. That is, the detection limit of the detection platform for the cocaine was approximately 1.5 mM, and the detection platform exhibited high sensitivity for identifying the cocaine.

According to the result of FIG. 8C, recognition accuracy of the detection platform for the ketamine at the concentration of 0.8 mM to 6 mM was 100%, while recognition accuracy for the ketamine at the concentration of 0.4 mM was 50.0%. That is, the detection limit of the detection platform for the ketamine was approximately 0.8 mM, and the detection platform exhibited high sensitivity for identifying the ketamine.

According to the result of FIG. 8D, recognition accuracy of the detection platform for the methamphetamine at the concentration of 1.6 mM to 20 mM was 100%, while recognition accuracy for the methamphetamine at the concentration of 0.8 mM was 66.7%. That is, the detection limit of the detection platform for the methamphetamine (or amphetamine type) was approximately 1.6 mM, and the detection platform exhibited high sensitivity for identifying the methamphetamine (or the amphetamine type).

According to the result of FIG. 8E, recognition accuracy of the detection platform for the heroine at the concentration of 1.6 mM to 20 mM was 100%, while recognition accuracy for the heroin at the concentration of 0.8 mM was 83.3%. That is, the detection limit of the detection platform for the heroin was approximately 1.6 mM, and the detection platform exhibited high sensitivity for identifying the heroin.

According to the result of FIG. 8F, recognition accuracy of the detection platform for the glucose at the concentration of 0.5 mM to 20 mM was 100%, and there was no false-positive result.

In the experimental example, since the detection platform exhibited the high sensitivity for identifying the 4-chloroethcathinone (the cathinones), the cocaine, the ketamine, the methamphetamine (or the amphetamine type), and the heroin, the detection platform of the experimental example may accurately identify abused drugs and effectively reduce the probability of false identification.

### [Experimental Example 9]: Analysis of Smuggled Samples by Using a Detection Platform

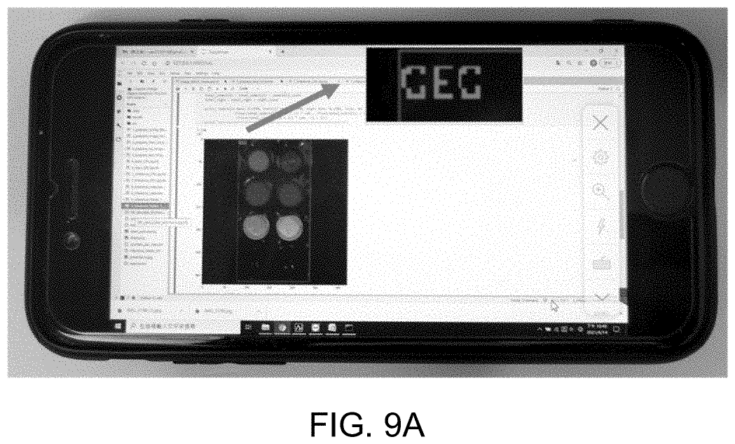

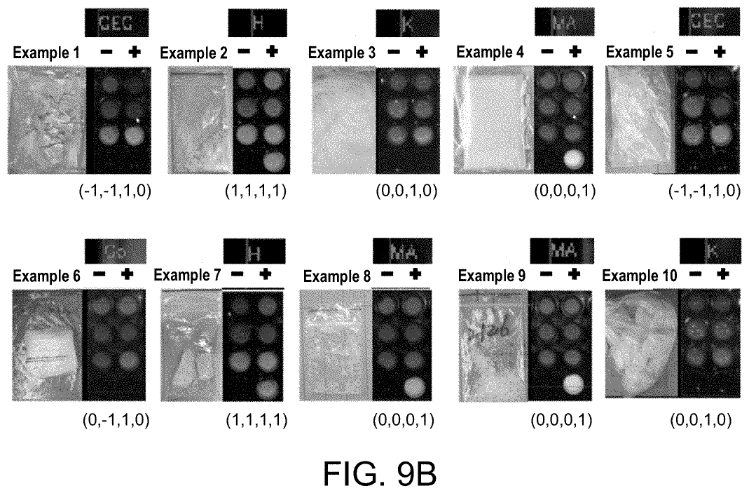

In the experimental example, ten smuggled samples (i.e. Example 1 to Example 10) in the real world were analyzed by using the platform system of deep learning drug detection (i.e. the detection platform) of Experimental example 6 to identify abused drug in the smuggled samples. Specifically, 5 to 10 mg of the samples were dissolved in 250 μl of ultrapure water first to make an analyte. Next, 50 μl of the analyte was added into the first three second grooves among the four second grooves of the sensing array, respectively, and 50 μl of the ultrapure water was added into the first three first grooves among the four first grooves of the sensing array so that the analyte may react with the gold nanoclusters, the carbon quantum dots, and the silver nanoclusters in the second grooves, respectively. Next, after 10 μl of the analyte and the Marquis reagent reacted for 2 minutes, an alkaline solution was added for neutralization. Then, 50 μl of the neutralized solution and 200 μl of a phosphate buffer solution were mixed to obtain a mixed solution. The mixed solution was added into the fourth second groove among the four second grooves of the sensing array. Next, after 10 μl of the ultrapure water and the Marquis reagent reacted for 2 minutes, the alkaline solution was added for neutralization. Then, 50 μl of the neutralized solution and 200 μl of the phosphate buffer solution were mixed to obtain a mixed solution. The mixed solution was added into the fourth first groove among the four first grooves of the sensing array. Next, under ultraviolet light with a wavelength of 365 nm, a detection result was obtained. Fluorescence matrix images of the detection result were captured by using the smartphone (e.g. iPhone 6S), and the deep learning computer set in advance was instantly connected for transmission. The images were cropped to remove the background to leave the images of only the sensing array. The pixel size of the images was adjusted to 320×320 pixels by using the computer. Lastly, automatic identification was performed through the connection of the smartphone and the deep learning computer, and a fluorescence detection code (W1, X1, Y1, Z1) of the analyte was compared to the known codes of the abused drugs to automatically identify which abused drug included in the analyte. The results are shown in FIG. 9A and FIG. 9B.

In the experimental example, the smuggled samples included Example 1 to Example 10. In addition, according to the results of Experimental example 5 and Experimental example 7, the identification code and the fluorescence detection code of the deep learning platform for the cathinones were “CEC” and (−1, −1, 1, 0), respectively, the identification code and the fluorescence detection code of the deep learning platform for the cocaine are “Co” and (0, −1, 1, 0), respectively, the identification code and the fluorescence detection code of the deep learning platform for the ketamine were “K” and (0, 0, 1, 0), respectively, the identification code and the fluorescence detection code of the deep learning platform for the methamphetamine were “MA” and (0, 0, 0, 1), respectively, the identification code and the fluorescence detection code of the deep learning platform for the heroin were “H” and (1, 1, 1, 1), respectively, and the identification code and the fluorescence detection code of the deep learning platform for the glucose were “G” and (0, 0, 0, 0), respectively.

According to the results of FIG. 9A and FIG. 9B, since the identification code and the fluorescence detection code of the deep learning platform for Example 1 were “CEC” and (−1, −1, 1, 0), respectively, it may be automatically identified that Example 1 may include the cathinones. Since the identification code and the fluorescence detection code of the deep learning platform for Example 2 were “H” and (1, 1, 1, 1), respectively, it may be automatically identified that Example 1 may include the heroin. Since the identification code and the fluorescence detection code of the deep learning platform for Example 3 were “K” and (0, 0, 1, 0), respectively, it may be automatically identified that Example 3 may include the ketamine. Since the identification code and the fluorescence detection code of the deep learning platform for Example 4 were “MA” and (0, 0, 0, 1), respectively, it may be automatically identified that Example 4 may include the amphetamine. Since the identification code and the fluorescence detection code of the deep learning platform for Example 5 were “CEC” and (−1, −1, 1, 0), respectively, it may be automatically identified that Example 5 may include the cathinones. Since the identification code and the fluorescence detection code of the deep learning platform for Example 6 were “Co” and (0, −1, 1, 0), respectively, it may be automatically identified that Example 6 may include the cocaine. Since the identification code and the fluorescence detection code of the deep learning platform for Example 7 were “H” and (1, 1, 1, 1), respectively, it may be automatically identified that Example 7 may include the heroin. Since the identification code and the fluorescence detection code of the deep learning platform for Example 8 were “MA” and (0, 0, 0, 1), respectively, it may be automatically identified that Example 8 may include the amphetamine. Since the identification code and the fluorescence detection code of the deep learning platform for Example 9 were “MA” and (0, 0, 0, 1), respectively, it may be automatically identified that Example 9 may include the amphetamine. Since the identification code and the fluorescence detection code of the deep learning platform for Example 10 were “K” and (0, 0, 1, 0), respectively, it may be automatically identified that Example 10 may include the ketamine.

In the experimental example, after the abused drug in the smuggled samples were identified with the detection platform, the abused drug in Example 1 to Example 10 were further analyzed with gas chromatography-mass spectrometry (GC-MS). According to the results of analysis of gas chromatography-mass spectrometry, it may be obtained that Example 1 was the 4-chloromethcathinone (the cathinones), Examples 2 was the heroin, Example 3 was the ketamine, Example 4 was the amphetamine, Example 5 was 4′-chloro-α-pyrrolidinopropiophenone (the cathinones), Example 6 was the cocaine, Example 7 was the heroin, Example 8 was the methamphetamine, Example 9 was the methamphetamine, and Example 10 was the ketamine. Therefore, it may be obtained that the abused drug identified by the detection platform were the same as the results of the analysis of gas chromatography-mass spectrometry. Accordingly, it may be proved that the detection platform and the method for detecting the abused drug of the experimental examples may rapidly and accurately identify abused drugs and may serve as a rapid screening tool at an anti-drug scene.

In summary of the above, in the detection platform and the method for detecting the abused drug of the embodiments of the disclosure, by providing the gold nanoclusters, the carbon quantum dots, the silver nanoclusters, and the mixed solution after reaction with the Marquis reagent in the sensing array, the detection platform may exhibit the high selectivity and high sensitivity for identifying 4-chloroethcathinone (cathinone), cocaine, ketamine, heroin, and methamphetamine (amphetamine). Accordingly, the detection platform and the method for detecting the abused drug of the embodiment may rapidly and accurately identify abused drugs and effectively reduce the probability of false identification. In addition, through connection between the image and transmission tool and the remote workstation and automatic identification, in the detection platform and the method for detecting the abused drug of the embodiment, the abused drug may be automatically and accurately identified through a computer of the remote station, and the disclosure may serve as a rapid screening tool at an anti-drug scene.

Although the disclosure has been described with reference to the above embodiments, they are not intended to limit the disclosure. It will be apparent to one of ordinary skill in the art that modifications to the described embodiments may be made without departing from the spirit and the scope of the disclosure. Accordingly, the scope of the disclosure will be defined by the attached claims and their equivalents and not by the above detailed descriptions.

What is claimed is:

1. A detection platform adapted to detect an abused drug in a sample, the detection platform comprising:
a sensing array comprising:
a reaction container having a plurality of first grooves and a plurality of second grooves; and
a plurality of gold nanoclusters, a plurality of carbon quantum dots, a plurality of silver nanoclusters, and a mixed solution after reaction with a Marquis reagent respectively arranged in corresponding first grooves and corresponding second grooves,
wherein, under ultraviolet light, the gold nanoclusters have first fluorescence, the carbon quantum dots have second fluorescence, and the silver nanoclusters have third fluorescence,
wherein, after the abused drug reacts with the gold nanoclusters, the carbon quantum dots, and the silver nanoclusters in the second grooves, respectively, and the mixed solution after the abused drug reacting with the Marquis reagent is added into the second groove, a detection result is obtained;
an image and transmission tool configured to capture an image of the detection result, be connected to a remote workstation, and transmit the image to the remote workstation; and
the remote workstation configured to analyze the image and automatically identify the abused drug.

2. The detection platform according to claim 1, wherein, in response to the abused drug being cathinone, the abused drug causes the first fluorescence to be quenched, the second fluorescence to be quenched, and the third fluorescence to be enhanced, and no fluorescence polymer particles are generated in the mixed solution after the abused drug reacting with the Marquis reagent.

3. The detection platform according to claim 1, wherein, in response to the abused drug being cocaine, the abused drug does not cause the first fluorescence to be quenched, the abused drug causes the second fluorescence to be quenched and causes the third fluorescence to be enhanced, and no fluorescence polymer particles are generated in the mixed solution after the abused drug reacting with the Marquis reagent.

4. The detection platform according to claim 1, wherein, in response to the abused drug being ketamine, the abused drug does not cause the first fluorescence to be quenched, the abused drug does not cause the second fluorescence to be quenched, the abused drug causes the third fluorescence to be enhanced, and no fluorescence polymer particles are generated in the mixed solution after the abused drug reacting with the Marquis reagent.

5. The detection platform according to claim 1, wherein, in response to the abused drug being amphetamine type, the abused drug does not cause the first fluorescence to be quenched, the abused drug does not cause the second fluorescence to be quenched, the abused drug does not cause the third fluorescence to be enhanced, and fluorescence polymer particles with fourth fluorescence are generated in the mixed solution after the abused drug reacting with the Marquis reagent.

6. The detection platform according to claim 1, wherein, in response to the abused drug being heroin, the abused drug causes the first fluorescence to be enhanced, the abused drug causes the second fluorescence to be enhanced, the abused drug causes the third fluorescence to be enhanced, and fluorescence polymer particles with fourth fluorescence are generated in the mixed solution after the abused drug reacting with the Marquis reagent.

7. The detection platform according to claim 1, wherein a sensing mechanism of the gold nanoclusters for the abused drug is electron transfer, a sensing mechanism of the carbon quantum dots for the abused drug is electron transfer, a sensing mechanism of the silver nanoclusters for the abused drug is aggregation-induced enhancement, and a sensing mechanism of the Marquis reagent for the abused drug is whether to generate fluorescence polymer particles.

8. A method for detecting an abused drug, wherein the method is adapted to detect the abused drug in a sample, and the method comprises:
providing the detection platform according to claim 1;
adding the sample into the second grooves of the sensing array so that the abused drug reacts with the gold nanoclusters, the carbon quantum dots, and the silver nanoclusters, respectively;
adding the mixed solution after the abused drug reacting with the Marquis reagent into the second groove;
obtaining the detection result with illumination of the ultraviolet light;
capturing the image of the detection result by using the image and transmission tool, wherein the image and transmission tool is connected to a remote workstation and transmits the image to the remote workstation; and
analyzing the image by using the remote workstation and automatically identifying the abused drug.

9. The method for detecting the abused drug according to claim 8, wherein before obtaining the detection result with the illumination of the ultraviolet light, the method further comprises:
adding a control group without the abused drug into the first grooves of the sensing array so that the control group reacts with the gold nanoclusters, the carbon quantum dots, and the silver nanoclusters, respectively; and
adding the mixed solution after the control group reacting with the Marquis reagent into the first groove.

10. The method for detecting the abused drug according to claim 8, wherein analyzing the image by using the remote workstation and automatically identifying the abused drug comprise:
cropping a background of the image; and
adjusting a pixel size of a cropped image.

11. The method for detecting the abused drug according to claim 8, wherein the abused drug comprises cathinones, cocaine, ketamine, amphetamine type, or heroin, a detection limit of the detection platform for the cathinones (e.g. 4-chloroethcathinone) is 2 mM, a detection limit of the detection platform for the cocaine is 1.5 mM, a detection limit of the detection platform for the ketamine is 0.8 mM, a detection limit of the detection platform for the amphetamine is 1.6 mM, and a detection limit of the detection platform for the heroin is 1.6 mM.
